(19) United States
(12) Patent Application Publication
(10) Pub. No.: US 2021/0177432 A1
(43) Pub. Date: Jun. 17, 2021

(54) Smart Tourniquet

(71) Applicant: Fiomet Ventures, Inc., Cincinnati, OH (US)

(72) Inventor: Scott Rapp, Cincinnati, OH (US)

(21) Appl. No.: 17/163,353

(22) Filed: Jan. 30, 2021

Related U.S. Application Data
(63) Continuation-in-part of application No. 15/289,071, filed on Oct. 7, 2016, now Pat. No. 10,973,413.
(60) Provisional application No. 62/969,067, filed on Feb. 1, 2020.
(60) Provisional application No. 62/238,522, filed on Oct. 7, 2015.

(51) Int. Cl. A61B 17/135; A61F 7/02; G16H 40/67; G16H 50/50; G16H 15/00

(52) U.S. Cl. CPC A61B 17/1355; A61F 7/02; A61B 2017/00876; G16H 50/50; G16H 15/00; G16H 40/67

(57) ABSTRACT

A multilayer smart tourniquet applied about a wound environment or injured tissue. The smart tourniquet can be supplied with sensors and micro heating and cooling units.

This Application claims the benefit of Provisional Application 62/969,067—Wound Treatment Devices—filed on Feb. 1, 2020 and priority to U.S. application Ser. No. 15/289,071—Advanced Compression Garments and Systems—, filed Oct. 7, 2016 that claimed the benefit of U.S. Provisional Application 62/238,522, filed on Oct. 7, 2015.

### BACKGROUND OF THE INVENTION

### A. Field of the Invention

The current smart tourniquet utilizes smart technology and compression structures that can assist in controlling venous and arterial bleeding. The tourniquet can also supply pressure to a soft tissue defect or wound. Use of smart technology provides real-time data sensed by pressure and other biometrics to the user. It is believed that use of the present smart tourniquet can reduce morbidity associated with post injury soft tissue and lessen mortality rates from penetrating injuries.

Time is the most critical factor from the onset of injury during battle to medical intervention. It has been estimated that over 90% of deaths in soldiers occur before they are evaluated at a stand-alone medical facility. Further, it is estimated that over 25% of these deaths may be preventable¹. Historically, extremity hemorrhage is responsible for over 8% of all battlefield deaths spanning from the Civil War until the 1990s. This led to multiple wings of the military branches to explore utilization of portable devices that can address uncontrolled hemorrhage from penetrating or blast injuries.

To combat preventable morbidity and mortality in extremity injuries, the use of tourniquets as a hemostatic intervention has taken various similar forms over the last few centuries. First implemented by Morel in 1674², the technology has largely gone unchanged since with little overall improvement to design. In 1873, Esmarch described use of a rubber bandage to exsanguinate the limb and application of a tourniquet made from rubber tubing³. It was reported that even as recently as 2001, tourniquet use was still limited to available resources such as bandanas, sticks, belts and other nearby materials'. The basic premise for the tourniquet is to apply pressure over an axial or named artery, where twisting of a band about the soft tissue creates local compression and ischemia.

Medical professionals have been critical of tourniquet use due to multiple problems with its technology leading to the lack of universal adoption and acceptance in trauma and critical care even today. Focal application over a penetrating injury may not address the physical location of the hemorrhage. Arterial injuries often result in retraction of the vessel with migration quite proximally to the site of the injury. Tourniquet application in these scenarios may lead to venous hypertension as the veins are compressed. At the same time, unaffected collateral arteries may continue to remain patent despite external compression due to arterial lumen pressure exceeding the topical pressure applied. Blood flow may continue to travel past the tourniquet into the distal extremity. However, since the venous system is more easily compressed, returning blood flow may not be able to travel back through the tourniquet and thus resulting in extremity venous hypertension as blood volume increases distal to the tourniquet. Clinically this leads to increased lumen pressure in the venous system which gradually builds up over time. Once the lumen pressure in the veins exceeds the tourniquet compression forces, further hemorrhage or exsanguination can occur. Venous bleeding can appear or mimic arterial bleeding in nature and blood loss can be accelerated. Precise application of pressure over a damaged artery can be difficult on the battle field.

Prolonged ischemia times greater than 2 hours leads to observable and irreversible apoptosis in human cells under microscopy. Excessive pressure over a nerve can cause irreversible damage and chronic pain syndromes. Greater than 6 hours of tourniquet application usually leads to unsalvageable soft tissue loss resulting in amputation. Improper tourniquet application to a leg can be managed with a less morbid BKA (Below the Knee Amputation) or could require an AKA (Above the Knee Amputation). Median survival rates at 5 years have as low as 22.5% with an AKA and 37.8% with a BKA⁴. An AKA requires utilization of more resources and time for rehabilitation. It appears that post injury tissue preservation can improve multiple quality of life factors.

Further, to complicate tourniquet application, it was thought that letting the tourniquet down periodically would reduce these above adverse effects. However, re-perfusion injury from harmful ischemic metabolites and resultant venous hypertension can lead to further tissue loss. Venous thrombosis can limit blood return and increase hypertension.

In early 2000s, the Joint Combat Casualty Research (JCCR) provided independent research on the value of tourniquet utilization and increased survival. By 2005-2006, all Special Operations units were deployed with tourniquets. Newly designed tourniquets became commercially available and in 2008, the World War II tourniquet design was finally abandoned. The first widespread branded tourniquet was the CAT tourniquet or Combat Application Tourniquet. The CAT device is composed of durable nylon band around 3 cm across with a rigid rod fit through a loop to twist for tightening. Hook and loop fastener was added to prevent loosening. A second version called SAM Junctional Tourniquet (SJT) was implemented for junctional injuries to limbs. Results were favorable in the field. The 75th Ranger Regiment saw mortality rates drop to 0% for hemorrhage and saw preventable deaths account for just 3 percent of its fatalities, compared to 24 percent of all U.S. military fatalities⁵.

### B. Description of the Previous Art

Any discussion of references cited herein merely summarizes the disclosures of the cited references. Applicant makes no admission that any cited reference or portion thereof is relevant prior art. Applicant reserves the right to challenge the accuracy, relevancy and veracity of the cited references.

Publications or presentations that may indicate a state-of-art include:

1. US Army Institute of Surgical Research. Stopping the Bleed. How army surgeons brought tourniquets back into the medical mainstream. Excerpted from document presented Feb. 2, 2018.

2. Welling D R, McKay P L, Rasmussen T E, Rich N M. A brief history of the tourniquet. J Vasc Surg. 2012 January; 55(1):286-90.

3. Esmarch F. Ueber Kunstliche Bluterlee bei Operationen. Samml Klin Vortr 1873; 58:373-384.

4. Aulivola B, Hile C N, Hamdan A D, Sheahan M G, Veraldi J R, Skillman J J, Campbell D R, Scovell S D, LoGerfo F W, Pomposelli F B Jr. Major lower extremity amputation: outcome of a modern series. Arch Surg. 2004 April; 139(4):395-9; discussion 399.

5. Kotwal R S, Montgomery H R, Kotwal B M, Champion H R, Butler F K Jr, Mabry R L, Cain J S, Blackbourne L H, Mechler K K, Holcomb J B. Eliminating preventable death on the battlefield. Arch Surg. 2011 December; 146(12):1350-8.

Patents that may indicate a state-of-art include: 1) U.S. Pat. No. 7,892,253—Esposito, et al. discloses a tourniquet and method of use (the CAT tourniquet); 2) U.S. Pat. No. 9,492,177—Saunders, et al. discloses a junctional tourniquet places focal pressure on the core body when vessels are injured proximal to the limb; 3) U.S. Pat. No. 4,469,099—McEwen discloses a pneumatic tourniquet; 4) U.S. Pat. No. 4,479,494—McEwen discloses an adaptive pneumatic tourniquet; 5) U.S. Pat. No. 5,439,477—McEwen discloses a tourniquet apparatus for applying minimum effective pressure; 6) U.S. Pat. No. 5,556,415—McEwen discloses a physiologic tourniquet for intravenous regional anesthesia; and 7) U.S. Pat. No. 5,855,589—McEwen discloses a physiologic tourniquet for intravenous regional anesthesia.

### SUMMARY OF THE INVENTION

Among other things, the current smart tourniquet can supply adjustable or constant pressures to a human wound. A communications module is detachable from the tourniquet. The communications module includes a transmitter or transceiver adapted to communicate with a Cloud server or computer, other computer such as a mobile computing device or a personal computer or any type of computing device remote from the communications module. Remote devices can be utilized to monitor or control pressures applied by the current smart tourniquet to cranial, venous, arterial or soft tissue wounds.

The current device and system embodiments can include a custom fitted tourniquet that can provide real time biometric feedback from multiple different sensors. Therapeutic interventions such as hemostasis, compression, adaptive heating and cooling and improved wound care optimization are possible with the current smart tourniquet.

To address hemorrhage following injury, among other things, the tourniquet has a custom foaming capability to adapt to any contour irregularity. The foaming agents are able to expand into the defect or around the defect to provide focal compression and improved hemostasis. By providing diffuse pressure over a greater surface area in and around the wound, less pressure can be used to obtain hemostatic control. With penetrating injuries, the foam will expand into this area providing a more appropriate mold of the defect than conventional tourniquets. A bladder interposition made of polyurethane or other polymeric materials may be implemented to ensure a watertight pressure application and enhanced rigidity to the foamed dressing. The bladder can be attached to the outward side of the inner layer of the tourniquet. When indicated by medical parameters, the current tourniquet provides control of external compression (more or less) supplied to the wound environment.

Within the scope of the current invention, biometric sensors can be positioned in inner layer, the bladder or both. In select embodiments of the tourniquet, biometric sensors such as pressure sensors can be placed at multiple locations of the invention. Use of one or more pressure sensors can provide alerts indications and/or alerts of acceptable, excessive or insufficient pressures. Insufficient pressures may result in excessive hemorrhage, low perfusion pressures, ischemia, secondary hypoxia leading to soft tissue, muscle, organ and neuronal death. Excessive pressures may result in downstream ischemia, venous hypertension, and focal soft tissue, axonal and cellular compromise. A biometric sensor communicates with communications module adapted to communicate with a computer distinct from the communications module. Communications module includes a transmitter or a transceiver allowing wireless communications with a remote computer that provides real real-time calculation/determination of the biometric(s) sensed. Communication modules can also include a memory, a microprocessor or processor and software (computing component) for calculating the biometric measurements sensed by the biometric sensors and controlling the tourniquet's heat/cooling semiconductors. Depending on medical treatment parameters, the remote computer or the communications module's computer module can be used to calculate sensed biometrics and control the heat/cooling semiconductors. In accordance with the present invention, the computer or computer module can calculate biometrics, including but not limited to, pressure, temperature, blood pressure, lactate levels, pH, sodium levels, potassium level, glucose levels, apoptotic factors, nitric oxide (NO) and SVO2 levels sensed by their respective sensors.

In selected embodiments of the current invention, semiconductors, such as micro-Peltier coolers, can be included in the inner layer, the bladder, the foam or any combination to provide heating or cooling. The direction of the current flow through micro-semiconductors allows for either cooling or warming. Power for the semiconductors and other components of the current tourniquet can be provided by batteries, a connection with an alternating current power source or a radio frequency energy supply.

Within the ambit of the current invention, micro-semiconductors can be rigid or flexible or a combination thereof. The semiconductors can utilize metallic thermal interfaces to dissipate heat or cold. Select preferred embodiments of the micro-semiconductors can utilize ferromagnetic foils that transition between paramagnetic and ferromagnetic states and can drive a metallic shuttle generating a squeezed-film cooling effect during oscillation. The thermal interfaces can induce vibration of fluids in proximity with the thermal interfaces. Select embodiments of the thermal interfaces can be provided with carbon nanotubes that can decrease thermal interface resistance. The carbon nanotubes can be grown on gadolinium foil. Use of carbon nanotubes can improve performance of the semiconductors.

It is believed that use of micro-semiconductors allows the application of different voltages and current across two distinct semiconductors, thereby generating a “heat flux” of hot or cold across the semiconductors' interfaces. Direction of current crossing the interfaces changes the generated heat flux from hot to cold or cold to hot such that the change the direction of current can result in a 25 degree Celsius deferential of temperature proximate the interfaces.

The current smart tourniquet can be sized to fit about body parts such as head, limbs or torso. Tourniquets adapted to be fitted about a body part can include a zipper spanning the length of tourniquets to provide easier application to the wound environment.

Within the scope of the current invention, tourniquets with prefabricated lumens can be pulled over the wound environment. For select preferred embodiments of tourniquets with prefabricated lumens, straps, hook and loop fasteners, hooks and catches, zippers or any combination thereof can be utilized to further secure the tourniquet about the wound environment and improve the tourniquet's post application stability.

Within the ambit of the current invention, tourniquets without prefabricated lumens can be folded about the wound environment. In select preferred embodiments of tourniquets without prefabricated lumens, straps, hook and loop fasteners, hooks and catches, zippers or any combination thereof can be utilized to secure the tourniquet about the wound environment and improve the tourniquet's post application stability.

The current custom tourniquet also has applications in veterinary medicine. By way of illustration, the smart tourniquet can provide focal compression to the lower extremity of a horse.

An aspect of the present invention is that the current tourniquet can be utilized on varying surface morphologies of humans, bovine, canine, equine, feline or zoo animals, etc.

Another aspect of the present invention is to provide a multilayer tourniquet including biometric sensors.

Yet another aspect of the present invention is to provide a tourniquet utilizing micro heating and cooling units.

Still another aspect of the present invention is to provide a tourniquet including synthetic muscle.

Yet still another aspect of the present invention is to provide a detachable communications module including a touchscreen displaying interactive data that allows the user to control one or more layers of the multilayer tourniquet.

Another aspect of the present invention is to provide a tourniquet adapted for use with expanding foam to conform either the foam or the tourniquet to the wound environment.

Still another aspect of the present invention is to provide a receptacle distinct from the layers of the tourniquet for releasably holding the communications module.

Yet still another aspect of the present invention is to provide a tourniquet including therapeutic zones.

A preferred embodiment of the current invention can be described as a tourniquet adapted for use about a wound environment or an injured tissue; the tourniquet comprising: a) an inner layer proximate the wound environment or an injured tissue; the inner layer comprising; i) a plurality of sensors attached to the inner layer, wherein at least some of the sensors sense pressure; and ii) a first port adapted to receive and distribute a first portion of an expandable foam conforming to the wound environment; b) a bladder contacting an outward side of the inner layer; the bladder sandwiched between the inner layer and an outer layer of the tourniquet, wherein the bladder comprises a second port adapted to input a second portion of the expandable foam into the bladder, thereby causing the inner layer to conform to all or a portion of the wound environment; c) a plurality of semiconductors comprising thermal interfaces and ferromagnetic properties; the plurality of semiconductors connected to the inner layer, the foam or the bladder or a combination thereof, wherein relative to the positioning of each of the semiconductors about the wound environment and dependent on a direction of current flowing through the thermal interfaces, the current causes the semiconductor to heat or cool an area of the wound environment associated with the semiconductor's footprint; d) a receptacle distinct from the inner layer, the bladder and the outer layer; the receptacle adapted to releasably hold a communications module to the tourniquet, wherein the receptacle comprises a junction for the tourniquet's electrical connections; e) one or more of the inner layer, the bladder and the outer layer comprising: compositions, fabrics or combinations thereof causing the inner layer, the bladder or the outer layer to function as synthetic muscle when the compositions or fabrics are activated by on/off electric current such that micro-increments of compressive, static or decompressive forces are delivered to the wound environment or the injured tissue; f) a detachable communications module, distinct from the receptacle, comprising a housing adapted for connection with the junction; the housing comprising: i) a first face magnetically reciprocating with the junction; ii) an outward face comprising a touchscreen; and iii) a computer module comprising one or more of the following components: a microprocessor, a memory, a visual graphics unit, an audio unit, a transmitter or transceiver and a software for controlling the components, the tourniquet and displays by the touchscreen; g) the transmitter or transceiver adapted for wireless communications, via an available wireless cellular network and/or IEEE 802.11 protocol, with a Cloud server or other computer remote from the communications module; and h) circuitry providing connections between the communications module, the junction, the sensors and the semiconductors and a power source for the communications module.

Another preferred embodiment of the current invention can be described as a tourniquet adapted for use about a wound environment or an injured tissue; the tourniquet comprising: a) an inner layer comprising; i) a plurality of sensors attached to the inner layer, wherein at least some of the sensors sense pressure; and ii) a first port adapted to receive and distribute a first portion of an expandable foam conforming to the wound environment; b) heating and cooling semiconductors connected to the inner layer, wherein an activated semiconductor heats or cools its footprint on the wound environment or injured tissue; c) the inner layer and the outer layer comprising: compositions, fabrics or combinations thereof causing the inner layer or the outer layer to function as synthetic muscle when the compositions or fabrics are activated by on/off electric current and deliver micro-increments of compressive, static or decompressive forces to the wound environment or injured tissue; d) a receptacle, distinct from the layers, comprising a junction for the tourniquet's electrical connections, wherein the receptacle is adapted to releasably hold a communications module; e) a communications module, distinct from the receptacle, comprising a housing adapted for connection with the junction; the housing comprising: i) a first face magnetically reciprocating with the junction; ii) an outward face comprising a touchscreen; and iii) a computer module comprising one or more of the following components: a microprocessor, a memory, a visual graphics unit, an audio unit, a transmitter or transceiver adapted for wireless communications, via an available wireless cellular network and/or IEEE 802.11 protocol, with a Cloud server or other computer remote from the communications module and a software for controlling the components, the tourniquet and displays by the touchscreen; and f) circuitry providing connections between the communications module, the junction, the sensors and the semiconductors and a power source for the communications module.

Still another preferred embodiment of the current invention can be described as a detachable communications module adapted for use with a customizable multilayered tourniquet applied to a wound environment or injured tissue; the detachable communications module comprising a housing comprising: a) a first face adapted to reciprocate magnetically with an electrical connectivity junction of the customizable multilayered tourniquet; the junction proximate a receptacle distinct from the junction and layers of the customizable multilayered tourniquet; the receptacle adapted to releasably hold the housing; b) a computer module comprising one or more of the following components: a microprocessor, a memory, a visual graphics unit, an audio unit, a transmitter or transceiver and a software for controlling the components, the customizable multilayered tourniquet and displays by a touchscreen; the transmitter or transceiver adapted for wireless communications, via a wireless cellular network and/or IEEE 802.11 protocol, with a Cloud server or other computer remote from the communications module; c) circuitry providing connections between the communications module, the junction, a plurality of biometric sensors, synthetic muscle, a power source for the communications module and semiconductors comprising thermal interfaces and ferromagnetic properties, wherein relative to the positioning of each of the semiconductors about the wound environment or injured tissue and dependent on a direction of current flowing through the thermal interfaces, the current causes the semiconductor to heat or cool an area of the wound environment or injured tissue associated with the semiconductor's footprint; and d) the touchscreen positioned on a second side of the housing visible to a user; the touchscreen displaying interactive data associated with the wound environment or injured tissue to the user; the displayed data allowing the user to control operations of the semiconductors and the synthetic muscle contained in one or more layers of the customizable multilayered tourniquet.

It is the novel and unique interaction of these simple elements which creates the system within the ambit of the present invention. Pursuant to Title 35 of the United States Code, select preferred embodiments of the current invention follow. However, it is to be understood that the descriptions of the preferred embodiments do not limit the scope of the present invention.

### BRIEF DESCRIPTION OF THE DRAWINGS

FIG. 1 portrays a preferred embodiment of tourniquet (10) with some structures of tourniquet (10) cut away to show an example of a wound environment (200).

FIG. 2 discloses a section of a preferred inner layer of tourniquet (10).

FIG. 3 is an exploded perspective of inner layer (20) and communications module (100).

FIG. 4 portrays communications module (100), Cloud server or computer (400) and other computers (500) such as a mobile computing device (smart phone, tablet, etc.) or a personal computer or any other type of computing device remote from tourniquet (10).

FIG. 4a discloses interface (104) of communications module (100).

FIG. 5 is a perspective of outer layer (150) of tourniquet (10) positioned outward of inner layer (20) or bladder (80).

FIG. 6 is another perspective of outer layer (150) of tourniquet (10).

FIG. 7 is another perspective of outer layer (150) of tourniquet (10).

FIG. 8 is another perspective of outer layer (150) of tourniquet (10).

### DESCRIPTION OF THE PREFERRED EMBODIMENTS

Although the disclosure hereof is detailed to enable those skilled in the art to practice the invention, the embodiments published herein merely exemplify the present invention.

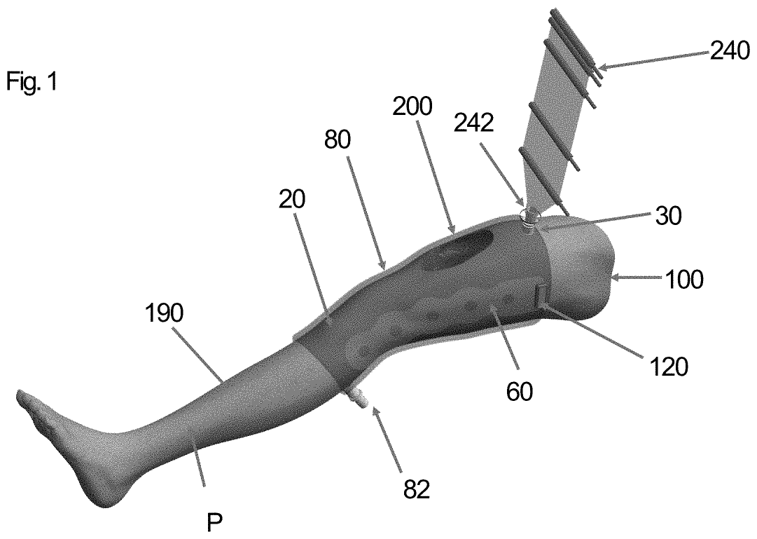

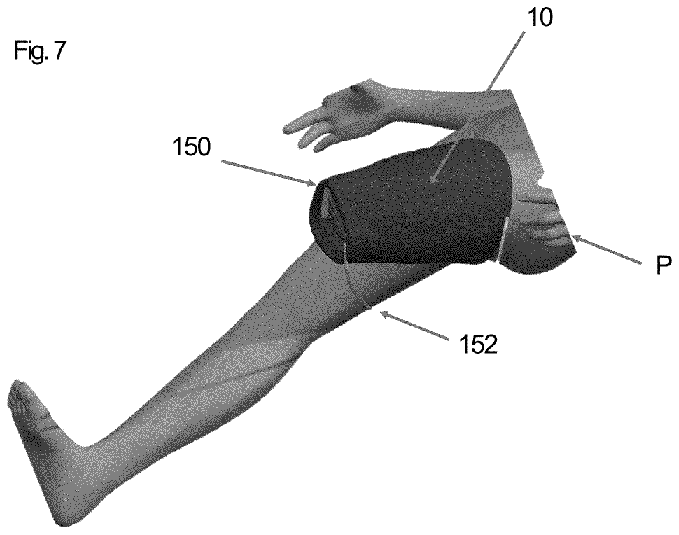

Preferred embodiments of Tourniquet (10) are disclosed in FIGS. 1-8.

FIG. 1 portrays a preferred embodiment of tourniquet (10) applied to patient's extremity (190) with some structures of tourniquet (10) cut away to show an example of a wound environment (200).

The current smart tourniquet (10) includes inner layer (20), outer layer (150) and communications module (100) connected to receptacle (120). Select preferred embodiments can be provided with an optional bladder (80) disposed outward from inner layer (20) and inward from outer layer (150).

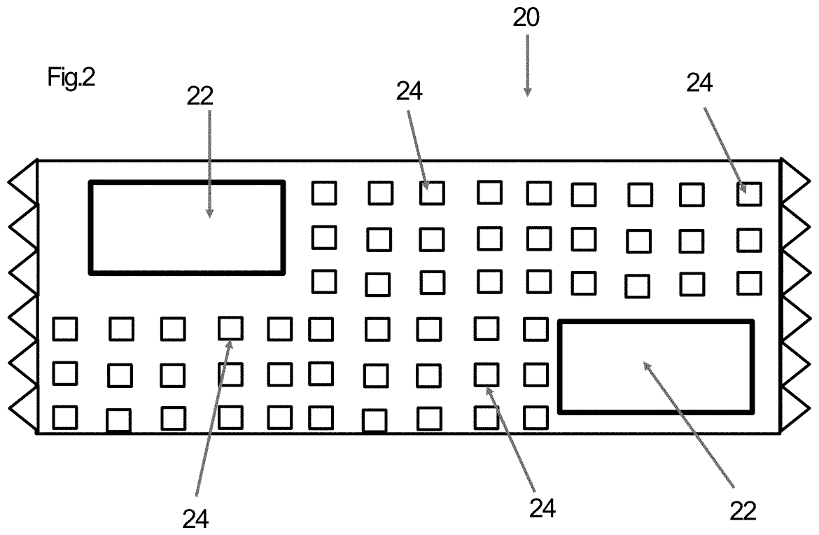

FIG. 2 discloses a section of a preferred inner layer of tourniquet (10). Inner layer (20) can be a flexible, soft and stretchable fabric constructed of one or more polymers or finely knit fabrics. By way of illustration, capillary vessel dilation can occur while absorbing heat away from the skin and inner layer (20) can be provided with a far infrared radiation fabric or bio ceramic-integrated fabric that can improve microcirculation about wound environment (200). Preferred embodiments of inner layer (20) can include hydrophobic adhesive polymers or equivalents that can assist in reducing movements of tourniquet (10) about wound environment (200) of extremity (190). Hydrophobic adhesive polymers or equivalents can be woven into or laminated onto inner layer (20). Inner layer (20) can include compositions impermeable to higher viscosity liquids and fluids. The inner layer (20) can be provided with one or more therapeutic zones (22) including one or more of the following: analgesic, anesthetic, anti-inflammatory, antimicrobial, hemostatic and vasoconstrictive agents, growth factors or any other medically beneficial composition combination thereof.

Select preferred embodiments of inner layer (20) are provided with semiconductors (24), e.g., micro-Peltier units. Although not shown in the Drawings, tourniquet (10) includes required circuitry interconnecting connecting semiconductors (24) to communications module (100). Current flow direction through semiconductors (24) controls whether semiconductors (24) generate heat or cooling. Footprints of semiconductors (24) can be less than 2 millimeters²to limit potential soft tissues injuries associated with wound environment (200) and nearby skin. Among other things, semiconductors (24) cooling and heating can assist in managing tissue swelling, contraction or secondary defect formation. When a patient is hypothermic, it may result in microcirculation collapse through higher vascular resistance and lead towards greater soft tissue ischemia or death. Heating the extremity may preserve perfusion to the effected extremity. Alternatively, cooling the extremity may assist in hemostasis and hemorrhage control. Power for semiconductors (24) and other components of the current tourniquet can be provided by batteries, a connection with an alternating current power source or a radio frequency energy supply. Among other things, heat/cool software can generate a heat map of temperatures within tourniquet (10) to better quantify therapeutic intervention required. Whether in the field or the hospital, it is believed that the selective control of temperatures generated by semiconductors (24) inside tourniquet (10) can improve medical outcomes for the patient. Inner layer (20) can be provided with a first port (30) adapted to couple with a device distinct from tourniquet (10) such as bag (240).

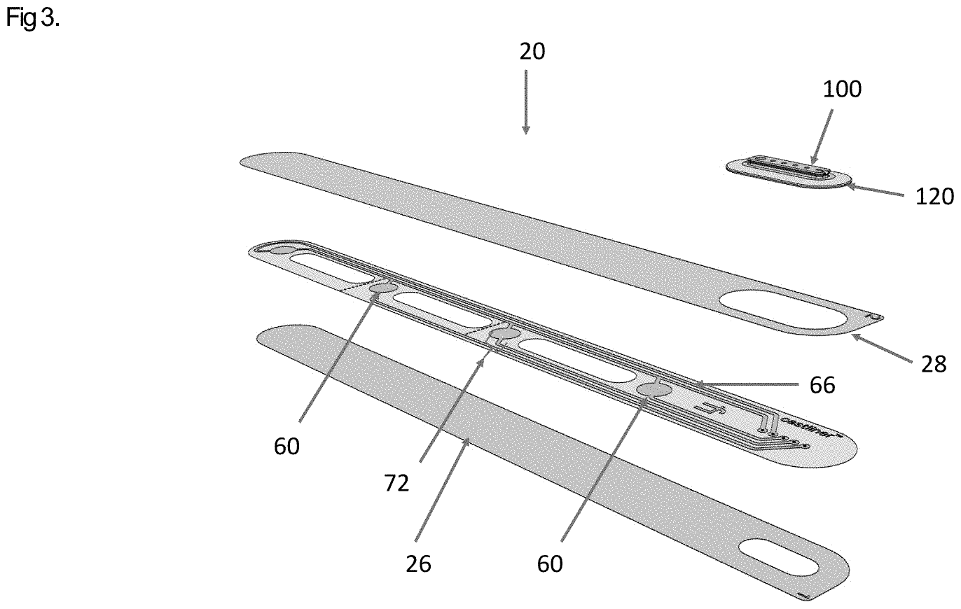

FIG. 3 is an exploded perspective of inner layer (20), optional bladder (80) and communications module (100). In preferred embodiments of the current invention, sensors (60) are associated with inner layer (20). Sensors (60) can be pressure sensing sensors. When engineering parameters require, sensors (60) can be sandwiched between first surface (26) and a second surface of inner layer (20) or bladder (80).

Sensors (60) can be printed onto inner layer (20) or include adhesive layers such as pressure sensitive adhesives (PSA), cement, heat activated polymers. Sensors (60) can be printed directly on inner layer (20) or be transferred on a TPU film through heat activation. For select embodiments, sensors (60) can be composed of electroactive inks (i.e copper, silver, platinum, carbon or combination), graphene or carbon nanotubes. One or more sensors (60) are capable of sensing biometrics, including but not limited to, pressure, temperature, blood pressure, lactate levels, pH, sodium levels, potassium level, glucose levels, apoptotic factors, nitric oxide and SVO2. Tourniquet (10) is provided with circuitry (66) connecting the one or more sensors (60) to communications module (100).

As previously indicated, bladder (80) can be disposed between inner layer (20) and outer layer (150). Bladder (80) is provided with second port (62) adapted to couple with a device distinct from tourniquet (10) such as bag (240).

Bladders (80) can include polymers such as polyurethane (PU) materials. When medical parameters require, bladder (80) can include compositions such as graphene or carbon nanotubes capable of acting as synthetic muscle when electric current is supplied to bladder (20). Use of synthetic muscle can provide increased compression, electronically controlled compression, or sequential compression of inner layer (20). Although not shown in the Drawings, tourniquet (10) is provided with circuitry adapted to connect synthetic muscle (80) to communications module (100).

Bag (240) includes nozzle (242) configured for engagement with port (82) of bladder (80). By way of illustration, bag (240) can include two compartmentalized liquids for creating silicone foam. When a seal separating bag's (240) compartments is broken, a silicon foam expansion reaction adapted to fill volume of wound environment (200) is catalyzed. The silicon foam expansion can be deployed about wound environment by extrusion directly into wound environment (200), into first port (30) of inner layer (20) or second port (82) of bladder (80) for subsequent filling of wound environment (200). Use of the current tourniquet (10) allows customized compression on any morphology to wound environment (200), i.e. large defect, amputation, etc. It has been discovered in situ set up of creating a foam layer for an underlying tissue defect can be accomplished in 10 minutes or less.

Tourniquet (10) is provided with receptacle (120) adapted to releasably hold communications module (100). Receptacle (120) can be distinct from inner layer (20) and composed of TPU materials, silicone, PU or other materials acceptable in the art. Receptacle (120) is provided with a junction for electrical connections and interconnected with some or all of the circuitry of tourniquet (10). In select preferred embodiments, receptacle (120) can be provided with one or more lips or rails to releasably hold communications module (100) to tourniquet (10). Among other things, removability of communications module (100) from tourniquet (10) allows cleaning or changing one or more layers (20, 80, 150) without damaging communications module (100).

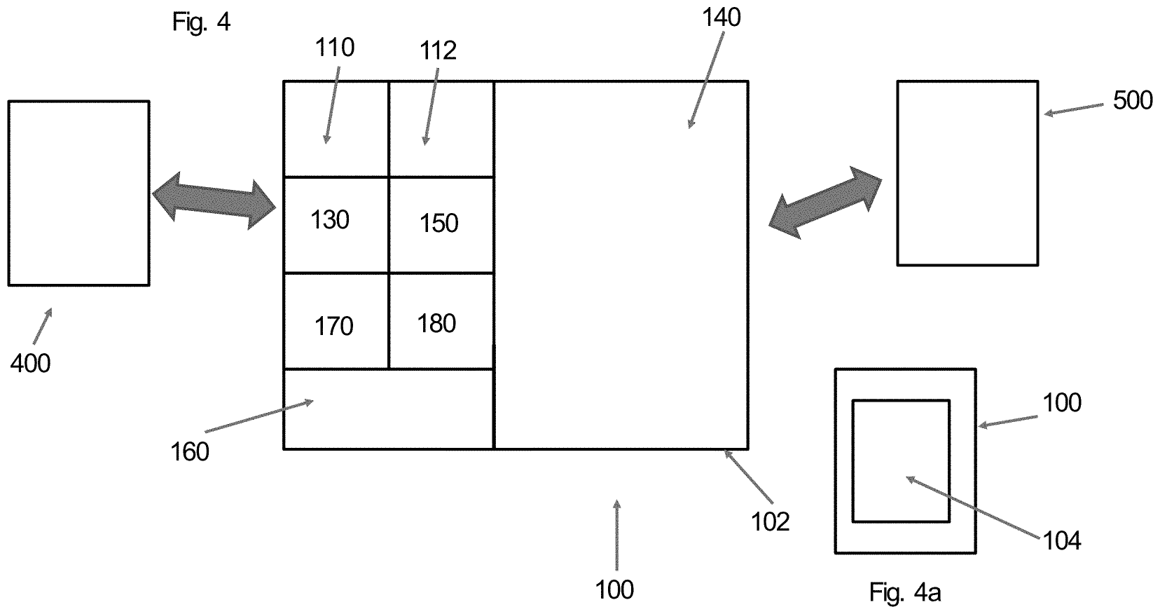

FIG. 4 portrays communications module (100), Cloud server or computer (400) and other computer (500) such as a mobile computing device (smart phone, tablet, etc.) or a personal computer or any other type of computing device remote from tourniquet (10). FIG. 4a discloses interface (104) of communications module (100).

Within the ambit of the current invention, communications module (100) can be provided with one or more of the following components: microprocessor (110), memory (112), visual graphics unit (130), touchscreen (140), audio unit (150), battery (160), transmitter or transceiver (170), circuitry interconnecting the components and software (180) for controlling the components. Housing (102) of communications module (100) can include interface (104) adapted to communicate with circuity (66) and junction of inner layer (10). Examples of potential interfaces (104) include pins, T-pins, magnets, metallic conductors or electroconductive materials. Touchscreen (102) can be an OLED or AMOLED touch screen (102) incorporated into an outward face of communications module (100). Within the scope of the current invention, tourniquet (10) can generate a visual or audible alarm when calculated preselected medical parameters are outside predetermined ranges.

Power sources for communications module (100) include but are not limited to rechargeable sources such as lithium ion, lithium iron phosphate, solid state, tab-less batteries or other usable power sources. Within the scope of the invention, the power source can be attached to housing (102). The rechargeable power sources can be detachable from housing (102) to engage the recharging energy supply.

Tourniquet (10) is adapted for wireless communications via any wireless network such as available cellular networks and/or IEEE 802.11 protocol at a frequency of 2.4 GHz (Wi-Fi or Bluetooth). Transmitter or transceiver (170) can communicate with a Cloud server or computer (400), other computer (500) such as a mobile computing device (smart phone, tablet, etc.) or a personal computer or any type of computing device remote from tourniquet (10). Among other things, devices remote from tourniquet (10) can be utilized to store data, calculate biometrics and/or control pressures applied by tourniquet (10) to the wound environment (200). By way of illustration, calculations of sensed biometric data can generate an alarm alerting the user of prolonged ischemic times associated with wound environment (200).

In other preferred embodiments of tourniquet (10), communications module (100) can also calculate biometrics and/or control pressures applied by tourniquet (10) to the wound environment (200). Within in the scope of the current invention, sensors (60) can be provided with transmitters or transceivers (72) for direct wireless communications with communications module (100) or other computing device remote from tourniquet (10).

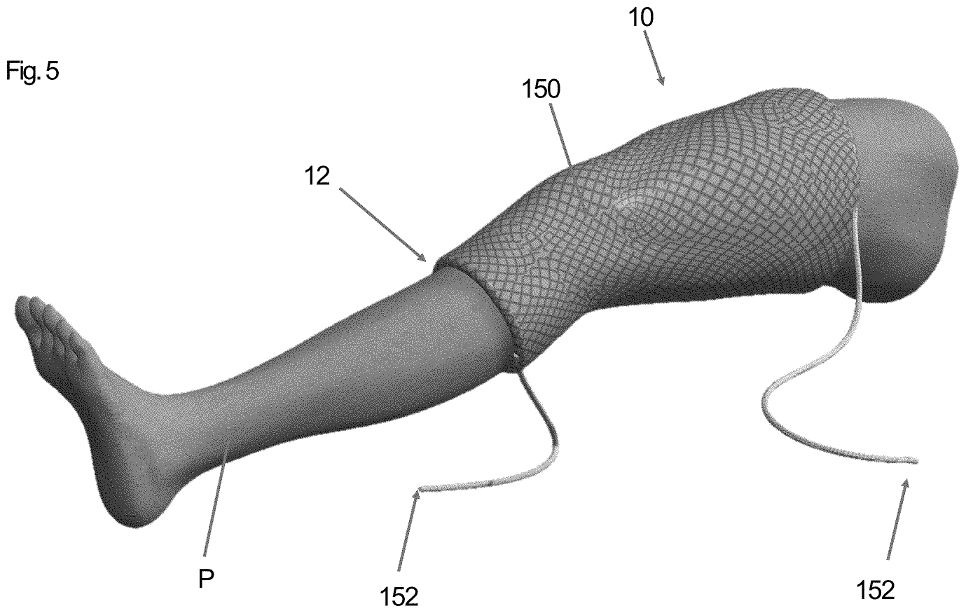

FIG. 5 is a perspective of outer layer (150) of tourniquet (10) positioned outward of inner layer (20) or bladder (80). Outer layer (150) can be composed of compressive materials and or woven fabrics capable of providing uniform distribution of pressure over soft tissue within lumen (12) of tourniquet (10). Cords (152) are incorporated with outer layer (150) and utilized to manually control compressive forces supplied by inner layer (20). In select preferred embodiments, outer layer (150) can include a dial apparatus (BOA® system) for cords (152) that can provide micro-control of compressive forces supplied to inner layer (20). The compressive materials can include far infrared technologies adapted to assist in improving microcirculation about wound environment (200). In select preferred embodiments of current tourniquet (10), on/off application of electric current to weaves of outer layer (150) can cause outer layer (150) to function as synthetic muscle and deliver micro-increments of compressive, static or decompressive forces to wound environment (200) or injured tissue. It is believed that outer layer's (150) provision of gentle compression assists with venous hemostasis and lessons venous hypertension. When medical parameters require, semiconductors (24) or other communications hardware can be incorporated into outer layer (150). Outer layer (150) can include components for communication with communications module (100).

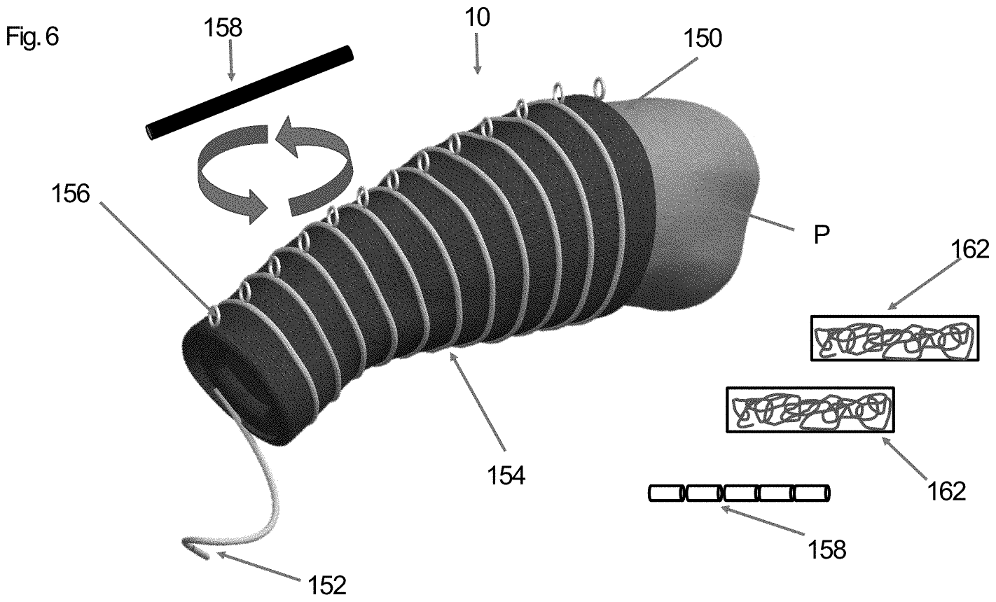

FIG. 6 is another perspective of outer layer (150) of tourniquet (10) applied to patient (P). Outer layer (150) can be provided with rings (154) with loops (156). This embodiment is intended for use with larger vessel diameter compression and intervention such as arteries and larger bore or diameter veins. In operation, one or more rods (158) are inserted through loops (156). Twisting or rotation of rods (158) can be performed (distal to proximal) to one or more loops (156) until the bleeding is stopped. Twisted rods (158) can be secured by hook and loop fasteners (162) attachable to outer layer (150). Rods (158) can be composed of lightweight materials (carbon fiber, nylons, nylon with glass, quarts tpt, etc). Rods (158) can be segmented and connected, multiple and/or collapsible for ease of storage. When rods (158) are not twisted, one or more rods (158) can provide segmental and rigid or semi-rigid fixation to the coronal plane to the patient's extremity (190). Rods (158) can be locked for providing rigid support along outer layer (150) of tourniquet (10). Segments of rods (158) can function as a splint. Select preferred embodiments of outer layer (150) can include a pocket for storage. Rings (154) can be composed of semi-rigid polymers or fabrics. One or more loops (156) can be provided with pressure sensors.

User interactive visual graphics of the calculated and correlated pressures, temperatures and sensed biometrics are visible on touch screen (140) of communications module (100) and/or computing devices (400, 500) remote from tourniquet (10). In select embodiments, the visual graphics can be color coded. Within the scope of the current invention, touchscreen (102) can display interactive data correlated and sensed by sensors (50) and semiconductors (60). The interactive, visualized, calculated and correlated data can be supplied to detachable communications module (100), cloud server (400) or computer remote (500) remote from communications module (100) or a combination thereof allowing the user of touchscreen (102) to control application of pressure to wound environment (200). Pressures supplied to wound environment (200) can also be controlled by cloud server (400) or computer remote (500) remote from communications module (100). Touchscreen (102) can also display interactive correlated data from biometric sensors (60) sensing one or more of pressure, temperature, blood pressure, lactate levels, pH, growth factors, hydrogen levels, sodium levels, potassium level, lactate levels, other electrolytes, cytokines, glucose levels, apoptotic factors, nitric oxide or SVO2. Touchscreen (102) can also display a pressure map or a heat map or both associated with wound environment (200) or injured tissue.

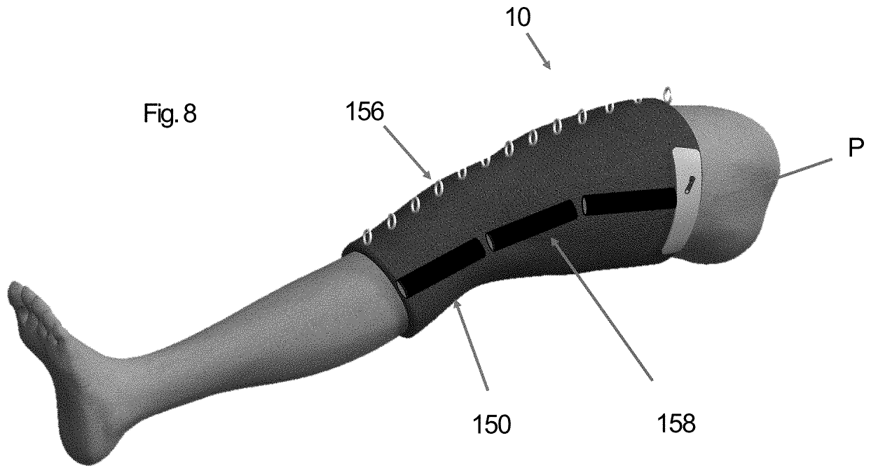

With reference to FIG. 8, another preferred embodiment of tourniquet (10) shows that outer layer (150) is provided with segmented rods (158) and loops (156). Segmented rods (158) can be utilized as one or more splints on one or more sides of outer layer (150). It is believed that his embodiment can be useful to support an extremity (190) of patient (P) having a femur fracture. Although not shown in the Drawings, tourniquet (10) can include a zipper spanning the length of tourniquet (10) for quicker application to an extremity. Other embodiments of tourniquet (10) can be provided with straps, hook and loop fasteners, hooks and catches, zippers or any combination to secure the tourniquet about the wound environment and improve the tourniquet's post application stability.

When medical parameters require, tourniquet (10) can be folded back on itself to generate additional pressure or compression. When required, one or more longitudinal ends of tourniquet (10) can be closed off for use in a stump compression. For a below the knee amputation, length of tourniquet (10) is adequate to extend beyond the knee. Tourniquet (10) is portable, flat, lightweight, transportable and is adapted for use in in hospital and field settings.

Select preferred embodiments of the current invention have been disclosed and enabled as required by Title 35 of the United States Code.

What is claimed is:

1) A tourniquet adapted for use about a wound environment or an injured tissue; the tourniquet comprising:
a) an inner layer proximate the wound environment or an injured tissue; the inner layer comprising;
i) a plurality of sensors attached to the inner layer, wherein at least some of the sensors sense pressure; and
ii) a first port adapted to receive and distribute a first portion of an expandable foam conforming to the wound environment;
b) a bladder contacting an outward side of the inner layer; the bladder sandwiched between the inner layer and an outer layer of the tourniquet, wherein the bladder comprises a second port adapted to input a second portion of the expandable foam into the bladder, thereby causing the inner layer to conform to all or a portion of the wound environment;
c) a plurality of semiconductors comprising thermal interfaces and ferromagnetic properties; the plurality of semiconductors connected to the inner layer, the foam or the bladder or a combination thereof, wherein relative to the positioning of each of the semiconductors about the wound environment and dependent on a direction of current flowing through the thermal interfaces, the current causes the semiconductor to heat or cool an area of the wound environment associated with the semiconductor's footprint;
d) a receptacle distinct from the inner layer, the bladder and the outer layer; the receptacle adapted to releasably hold a communications module to the tourniquet, wherein the receptacle comprises a junction for the tourniquet's electrical connections;
e) one or more of the inner layer, the bladder and the outer layer comprising: compositions, fabrics or combinations thereof causing the inner layer, the bladder or the outer layer to function as synthetic muscle when the compositions or fabrics are activated by on/off electric current such that micro-increments of compressive, static or decompressive forces are delivered to the wound environment or the injured tissue;
f) a detachable communications module, distinct from the receptacle, comprising a housing adapted for connection with the junction; the housing comprising:
i) a first face magnetically reciprocating with the junction;
ii) an outward face comprising a touchscreen; and
iii) a computer module comprising one or more of the following components: a microprocessor, a memory, a visual graphics unit, an audio unit, a transmitter or transceiver and a software for controlling the components, the tourniquet and displays by the touchscreen;
g) the transmitter or transceiver adapted for wireless communications, via an available wireless cellular network and/or IEEE 802.11 protocol, with a Cloud server or other computer remote from the communications module; and
h) circuitry providing connections between the communications module, the junction, the sensors and the semiconductors and a power source for the communications module.

2) The tourniquet of claim 1, wherein at least some of the plurality of sensors sense one or more of the following biometrics: pressure, temperature, blood pressure, lactate levels, pH, growth factors, hydrogen levels, sodium levels, potassium level, lactate levels, other electrolytes, cytokines, glucose levels, apoptotic factors or SVO2.

3) The tourniquet of claim 2 comprising an alarm, wherein the alarm is audible, visual or a combination thereof when the sensed biometric for a patient is outside of a predetermined range.

4) The tourniquet of claim 2, wherein the display on the housing's touchscreen was created by the communications module, the Cloud server, the other computer remote from the communications module or a combination thereof.

5) The tourniquet of claim 4, wherein the display portrays a pressure map or a heat map or both associated with the wound environment or the injured tissue.

6) The tourniquet of claim 4, wherein the inner layer comprises one or more therapeutic zones including one or more of the following compositions: analgesic, anesthetic, anti-inflammatory, antimicrobial, hemostatic and vasoconstrictive agents, growth factors or a combination thereof.

7) The tourniquet of claim 6, wherein one or more of the pressure sensors are incorporated into one or more loops associated with the outer layer.

8) The tourniquet of claim 4, wherein the thermal interfaces induce vibrations of fluids proximate the thermal interfaces and comprise carbon nanotubes adapted to decrease thermal interface resistance.

9) The tourniquet of claim 8, wherein up to about a 25 degree Celsius temperature differential is generated.

10) The tourniquet of claim 1, wherein the semiconductors are rigid or flexible or a combination thereof.

11) A tourniquet adapted for use about a wound environment or an injured tissue; the tourniquet comprising:
a) an inner layer comprising;
i) a plurality of sensors attached to the inner layer, wherein at least some of the sensors sense pressure; and
ii) a first port adapted to receive and distribute a first portion of an expandable foam conforming to the wound environment;
b) heating and cooling semiconductors connected to the inner layer, wherein an activated semiconductor heats or cools its footprint on the wound environment or injured tissue;
c) the inner layer and the outer layer comprising: compositions, fabrics or combinations thereof causing the inner layer or the outer layer to function as synthetic muscle when the compositions or fabrics are activated by on/off electric current and deliver micro-increments of compressive, static or decompressive forces to the wound environment or injured tissue;
d) a receptacle, distinct from the layers, comprising a junction for the tourniquet's electrical connections, wherein the receptacle is adapted to releasably hold a communications module;
e) a communications module, distinct from the receptacle, comprising a housing adapted for connection with the junction; the housing comprising:
i) a first face magnetically reciprocating with the junction;
ii) an outward face comprising a touchscreen; and
iii) a computer module comprising one or more of the following components: a microprocessor, a memory, a visual graphics unit, an audio unit, a transmitter or transceiver adapted for wireless communications, via an available wireless cellular network and/or IEEE 802.11 protocol, with a Cloud server or other computer remote from the communications module and a software for controlling the components, the tourniquet and displays by the touchscreen; and
f) circuitry providing connections between the communications module, the junction, the sensors and the semiconductors and a power source for the communications module.

12) The tourniquet of claim 11, wherein:
a) at least some of the plurality of sensors sense one or more of the following biometrics: pressure, temperature, blood pressure, lactate levels, pH, growth factors, hydrogen levels, sodium levels, potassium level, lactate levels, other electrolytes, cytokines, glucose levels, apoptotic factors, nitric oxide or SVO2; or
b) the inner layer comprises one or more therapeutic zones including one or more of the following compositions: analgesic, anesthetic, anti-inflammatory, antimicrobial, hemostatic and vasoconstrictive agents, growth factors or a combination thereof.

13) The tourniquet of claim 12 comprising a bladder layer sandwiched between the inner layer and an outer layer, wherein the bladder layer comprises a second port adapted to input a second portion of the expandable foam into the bladder layer, wherein the bladder layer causes the inner layer to conform to all or a portion of the wound environment;
14) The tourniquet of claim 13, wherein one or more of the pressure sensors are incorporated into one or more loops associated with the outer layer.

15) The tourniquet of claim 13, wherein the heating and cooling semiconductors are connected to the inner layer, the foam, the bladder or a combination thereof and comprise thermal interfaces and carbon nanotubes.

16) The tourniquet of claim 15 comprising one or more straps, hook and loop fasteners, hooks and catches, zippers or any combination thereof.

17) A detachable communications module adapted for use with a customizable multilayered tourniquet applied to a wound environment or injured tissue; the detachable communications module comprising a housing comprising:
a) a first face adapted to reciprocate magnetically with an electrical connectivity junction of the customizable multilayered tourniquet; the junction proximate a receptacle distinct from the junction and layers of the customizable multilayered tourniquet; the receptacle adapted to releasably hold the housing;
b) a computer module comprising one or more of the following components: a microprocessor, a memory, a visual graphics unit, an audio unit, a transmitter or transceiver and a software for controlling the components, the customizable multilayered tourniquet and displays by a touchscreen; the transmitter or transceiver adapted for wireless communications, via a wireless cellular network and/or IEEE 802.11 protocol, with a Cloud server or other computer remote from the communications module;
c) circuitry providing connections between the communications module, the junction, a plurality of biometric sensors, synthetic muscle, a power source for the communications module and semiconductors comprising thermal interfaces and ferromagnetic properties, wherein relative to the positioning of each of the semiconductors about the wound environment or injured tissue and dependent on a direction of current flowing through the thermal interfaces, the current causes the semiconductor to heat or cool an area of the wound environment or injured tissue associated with the semiconductor's footprint; and
d) the touchscreen positioned on a second side of the housing visible to a user; the touchscreen displaying interactive data associated with the wound environment or injured tissue to the user; the displayed data allowing the user to control operations of the semiconductors and the synthetic muscle contained in one or more layers of the customizable multilayered tourniquet.

18) The detachable communications module of claim 17, wherein the displayed data allows the user of the communications module to control:
a) delivery of micro-increments of compressive, static or decompressive forces to the wound environment or injured tissue; and/or
b) heating or cooling footprints supplied by one or more semiconductors to the wound environment or injured tissue.

19) The detachable communications module of claim 17, wherein the biometric sensors sense one or more of the following biometrics: pressure, temperature, blood pressure, lactate levels, pH, growth factors, hydrogen levels, sodium levels, potassium level, lactate levels, other electrolytes, cytokines, glucose levels, apoptotic factors, nitric oxide or SVO2.

20) The detachable communications module of claim 17, wherein the displayed data was created by the communications module, a Cloud server, another computer remote from the communications module or a combination thereof.

21) The detachable communications module of claim 18 comprising an audible, visual or a combination thereof alarm activated when the sensed biometric or a therapeutic for a patient is outside of a predetermined range.
